(19) United States
(12) Patent Application Publication
(10) Pub. No.: US 2026/0290590 A1
(43) Pub. Date: Sep. 24, 2026

(54) SELF-SUPERVISED TRAINING OF A MODEL ON TEMPORAL SEQUENCES OF PHYSIOLOGICAL MEASUREMENTS OF SUBJECTS

(71) Applicant: Ramot at Tel-Aviv University Ltd., Tel-Aviv (IL)

(72) Inventors: Lior WOLF, Tel-Aviv (IL); Noy LALZARY, Tel-Aviv (IL)

(73) Assignee: Ramot at Tel-Aviv University Ltd., Tel-Aviv (IL)

(21) Appl. No.: 19/477,503

(22) PCT Filed: Apr. 10, 2024
(86) PCT No.: PCT/IL2024/050356

§ 371 Date: Oct. 22, 2025

Related U.S. Application Data
(60) Provisional application No. 63/499,064, filed on Apr. 28, 2023.

(51) Int. Cl. G16H 50/20; G06N 3/088

(52) U.S. Cl. CPC G16H 50/20; G06N 3/088

(57) ABSTRACT

There is provided a method of predicting a state of a subject, comprising: feeding temporal sequences of physiological measurements into a machine learning (ML) model, and obtaining the state of the subject as an outcome of a second encoder of the ML model, wherein the ML model is trained by: for each sample dataset including temporal sequence of physiological measurements of a subject: feeding the sample dataset into the first encoder for obtaining a baseline latent representation, creating a copy latent representation(s), masking a time-interval(s) of each copy latent representation(s), and including the copy latent representation(s) and the baseline latent representation in a training dataset, training the ML model on the training dataset using a self-supervised approach, creating an updated training dataset by including a ground truth label for each sample dataset, and further training the second encoder on the updated training dataset using a supervised training approach.

### RELATED APPLICATION

This application claims the benefit of priority of U.S. Provisional Patent Application No. 63/499,064 filed on Apr. 28, 2023, the contents of which are incorporated herein by reference in their entirety.

### BACKGROUND

The present invention, in some embodiments thereof, relates to self-supervised training of a machine learning model and, more specifically, but not exclusively, to self-supervised training of a machine learning model on temporal sequences of physiological measurements of subjects.

Self-supervised learning is a machine learning paradigm where a model learns to make predictions about certain aspects of its input data without explicit supervision. The model may generate its own training labels from the input data itself, rather than relying on externally provided labels.

### SUMMARY

According to a first aspect, a method of predicting a state of a subject, comprises: feeding a dataset including a temporal sequence of physiological measurements of the subject into a machine learning (ML) model comprising a first encoder and a second encoder, and obtaining the state of the subject as an outcome of the second encoder, wherein the ML model is trained by: for each sample dataset of a plurality of sample datasets, wherein each sample dataset includes a sample temporal sequence of physiological measurements of the subject: feeding the sample dataset into the first encoder, obtaining a baseline latent representation as an outcome of the first encoder, creating at least one copy latent representation from the baseline latent representation, masking at least one time-interval of each at least one copy latent representation, and including the at least one copy latent representation and the baseline latent representation in a training dataset, in a first training phase, training the ML model including the first encoder and the second encoder on the training dataset using a self-supervised approach, creating an updated training dataset by including a ground truth label for each sample dataset in the training dataset, and in a second training phase, further training the second encoder on the updated training dataset using a supervised training approach.

According to a second aspect, a system for predicting a state of a subject, comprises: at least one processor executing a code for: feeding a dataset including a temporal sequence of physiological measurements of the subject into a machine learning (ML) model comprising a first encoder and a second encoder, and obtaining the state of the subject as an outcome of the second encoder, wherein the ML model is trained by: for each sample dataset of a plurality of sample datasets, wherein each sample dataset includes a sample temporal sequence of physiological measurements of the subject: feeding the sample dataset into the first encoder, obtaining a baseline latent representation as an outcome of the first encoder, creating at least one copy latent representation from the baseline latent representation, masking at least one time-interval of each at least one copy latent representation, and including the at least one copy latent representation and the baseline latent representation in a training dataset, in a first training phase, training the ML model including the first encoder and the second encoder on the training dataset using a self-supervised approach, creating an updated training dataset by including a ground truth label for each sample dataset in the training dataset, and in a second training phase, further training the second encoder on the updated training dataset using a supervised training approach.

According to a third aspect, a method of training a machine learning (ML) model, comprises: for each sample dataset of a plurality of sample datasets, wherein each sample dataset includes a temporal sequence of physiological measurements of the subject: feeding the sample dataset into a first encoder, obtaining a baseline latent representation as an outcome of the first encoder, creating at least one copy latent representation from the baseline latent representation, masking at least one time-interval of each at least one copy latent representation, and including the at least one copy latent representation and the baseline latent representation in a training dataset, in a first training phase, training the ML model including the first encoder and a second encoder on the training dataset using a self-supervised approach, creating an updated training dataset by including a ground truth label for each sample dataset in the training dataset, and in a second training phase, further training the second encoder on the updated training dataset using a supervised training approach.

In a further implementation form of the first, second, and third aspects, the ML model generates a prediction of a state of a subject from an input of a source dataset of an individual.

In a further implementation form of the first, second, and third aspects, further comprising: feeding the dataset into the first encoder to obtain a latent representation, feeding the latent representation into the second encoder, and obtaining the prediction of the state of the subject from the second encoder.

In a further implementation form of the first, second, and third aspects, further comprising during the second training phase: freezing at least one convolutional layer of the first encoder and/or freezing the first encoder, and adding a classification head on top of an output embedding of the ground truth.

In a further implementation form of the first, second, and third aspects, the first encoder is implemented as a one dimensional (1D) convolutional neural network CNN).

In a further implementation form of the first, second, and third aspects, the first encoder includes three convolution blocks, a max-pooling layer, and a dropout layer, and wherein each convolution block in the three convolution blocks comprises one convolution layer with a Gaussian Error Linear Unit (GELU) activation function.

In a further implementation form of the first, second, and third aspects, the second encoder is implemented as a transformer.

In a further implementation form of the first, second, and third aspects, during the first training phase, the ML model is trained according to a loss function comprising at least one temporal objective.

In a further implementation form of the first, second, and third aspects, during the first training phase, the ML model is trained according to a loss function comprising at least one contextual contrastive loss objective.

In a further implementation form of the first, second, and third aspects, further comprising, for each sample dataset: generating two sequences by cropping the at least one copy latent representation and the baseline latent representation, and adding a classification label indicating the ground truth label of the sample dataset, and feeding each of the two sequences to the second encoder for obtaining outcome embedding of the classification label, wherein during the first training phase the ML model further matches different latent representations of a sample dataset using contextual contrasting, such that at least one of: similarity between the outcome embedding of the classification labels of the two sequences is maximized, and similarity with the classification label is minimized.

In a further implementation form of the first, second, and third aspects, the two sequences are denoted as [Zᵢ]_{≥t}=[CLS, zᵢ¹, . . . , zᵢ^{t}] and [Z^{~}ᵢ]_{≤t}=[CLS, z^{~}ᵢ¹, . . . , z^{~}ᵢ^{t}], wherein CLS denotes the classification label, t denotes a random integer sampled from an interval (0,T−K], wherein K denotes a number of time-steps to be predicted as a pretext task, wherein Zᵢ=[zᵢ¹, zᵢ², . . . , zᵢ^{T}], Z^{~}ᵢ=[z^{~}ᵢ¹, z^{~}ᵢ², . . . , z^{~}ᵢ^{T}], Z^{~}_{I} denotes a copy of Zᵢ, Zᵢ=F(Xᵢ) wherein F denotes the first encoder, and Xᵢ denotes the sample dataset.

In a further implementation form of the first, second, and third aspects, the outcome embedding of the classification labels are denoted as eCLSi=T ([Zi]≤t) and e^{~}CLSi=T ([Z^{~}i]≤t), wherein e^{CLS}ᵢ, e^{~CLS}ᵢ∈R^{d} and d denotes a dimension of the second encoder.

In a further implementation form of the first, second, and third aspects, the first training phase comprises predicting by the ML model, at least one future sequence element by using a contrastive framework with a plurality of different linear projections, projecting the outcome embedding of the classification labels back to a dimension of the latent representations each predicting one of the future time intervals of the at least one copy latent representation and/or the baseline latent representation, and minimizing a dot product between each projected outcome and matching predicted future time interval while minimizing a dot product between each projected outcome and the future time intervals.

In a further implementation form of the first, second, and third aspects, an objective function for the first training phase is denoted as Lₚᵣₑ₋ₜᵣₐᵢₙ=λ₁(L_{TC}+L^{~}_{TC})+λ₂(L_{CC}+L^{~}_{CC}), where λ1 and λ2 are hyperparameters.

In a further implementation form of the first, second, and third aspects, an objective function for the second training phase is denoted as L_{fine-tune}=−L_{cce}(yᵢ, C(T[E(Xᵢ)]^{CLS})), where L_{cce} is a softmax function followed by a standard categorical cross-entropy loss and yᵢ∈{1, . . . , c} is an associated label.

In a further implementation form of the first, second, and third aspects, the dataset includes at least one of: ECG data from ECG signals obtained by electrodes on the subject, EEG data from EEG signals obtained by electrodes on the subject, EMG data from EMG signals obtained by electrodes on the subject.

In a further implementation form of the first, second, and third aspects, the dataset comprises a single-channel signal input.

In a further implementation form of the first, second, and third aspects, the state of the subject comprises an emotional state.

In a further implementation form of the first, second, and third aspects, the state of the subject comprises a physical state, wherein the ground truth of the physical state is obtained from measurements made by a sensor.

In a further implementation form of the first, second, and third aspects, the physical state is selected from: blood glucose level, blood oxygenation level, blood carbon dioxide level.

In a further implementation form of the first, second, and third aspects, the dataset is denoted as Xᵢ∈Rⁿ, where n denotes a multiplicative product of a frequency in Hertz and a length in seconds, and i denotes an index of a sample from which the dataset is extracted.

In a further implementation form of the first, second, and third aspects, the baseline latent representation and the masked copy latent representation are respectively denoted as Zᵢ, Z^{~}ᵢ∈R^{m×T}, wherein m denotes an output dimension of the first encoder and T denotes a number of the time-steps obtained from the input.

In a further implementation form of the first, second, and third aspects, masking comprises selecting a random proportion from a plurality of time-steps to be a starting point of a masked span of a selected length, and replacing each of the plurality of time-steps with zeros.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a block diagram of components of a system for training a machine learning model(s) on a training dataset and/or for inference by the machine learning model(s), in accordance with some embodiments of the present invention;

FIG. 2 is a flowchart of a method for training a machine learning model(s) for predicting a state of a subject from a temporal sequence of physiological measurements, in accordance with some embodiments of the present invention;

FIG. 3 is a flowchart of a method of predicting a state of a subject by a machine learning model(s) fed a temporal sequence of physiological measurements, in accordance with some embodiments of the present invention;

FIG. 4 is a schematic indicating an exemplary approach for pre-training of a ML model including a first encoder and a second encoder using temporal physiological data, in accordance with some embodiments of the present invention; and

FIG. 5 includes tables that present results of the experiments described herein, in accordance with some embodiments of the present invention.

### DETAILED DESCRIPTION

The present invention, in some embodiments thereof, relates to self-supervised training of a machine learning model and, more specifically, but not exclusively, to self-supervised training of a machine learning model on temporal sequences of physiological measurements of subjects.

An aspect of some embodiments of the present invention relates to systems, methods, computing devices, and/or code instructions (stored on a data storage device and executable by one or more hardware processors for predicting a state of a subject based on a dataset including temporal sequence of physiological measurements (e.g., obtained by a sensor), for example, electrocardiogram (ECG) electroencephalogram (EEG), and/or electromyography (EMG). The state of the subject may be, for example, an emotional state (e.g., happy, sad, anxious) and/or a physical state (e.g., blood glucose level, blood oxygen level). The dataset including the temporal sequence of physiological measurements of the subject is fed into a machine learning (ML) model that includes a first encoder and a second encoder. The dataset is fed into the first encoder. A latent representation obtained from the first encoder is fed into the second encoder. The state of the subject is obtained from the second encoder.

The ML model may be trained on multiple sample datasets obtained from one or more subjects. Each sample dataset includes a sample temporal sequence of physiological measurements of the respective subject. For each sample dataset: the sample dataset is fed into the first encoder. A baseline latent representation is obtained from the first encoder. A copy latent representation is created as a copy of the baseline latent representation. One or more time-intervals of the copy latent representation are masked. The copy latent representation and the baseline latent representation are included in a training dataset. In a first training phase, also referred to herein as a pre-training phase, the ML model including the first encoder and the second encoder is trained on the training dataset using a self-supervised approach. The ML model may be trained using a temporal objective and/or a contextual contrastive loss objection, optionally using a loss function that includes both objectives. An updated training dataset may be created by including a ground truth label for one or more sample datasets. Other sample datasets may remain without the ground truth labels. In a second training phase, also referred to herein as a fine-tuning phase, the second encoder may be further trained on the updated training dataset using a supervised training approach. The second encoder may be trained while the first encoder is frozen (i.e., not further trained).

At least one embodiment described herein addressed the technical problem of training a machine learning model for predicting a state of a subject from an input of a dataset that includes a temporal sequence of physiological measurements of the subject, where the amount of labelled data is insufficient for supervised training. Examples of datasets include ECG, EEG, and EMG. Examples of states of the subject include emotional and/or mental states (e.g., depression, happy, anxious), and/or physiological states measurable by a sensor (e.g., blood glucose level, blood oxygenation level, blood carbon dioxide level). At least one embodiment described herein improves the technical field of training a machine learning model for predicting a state of a subject from an input of a dataset that includes a temporal sequence of physiological measurements of the subject, where the amount of labelled data is insufficient for supervised training. At least one embodiment described herein improves upon prior approaches for training a machine learning model for predicting a state of a subject from an input of a dataset that includes a temporal sequence of physiological measurements of the subject, where the amount of labelled data is insufficient for supervised training.

It is noted that references to ECG are not meant to be necessarily limiting, but are provided as an example. References to ECG may be understood as referring to other temporal physiological signals, for example, EEG, EMG, and the like.

ECG is a safe, non-invasive, and cost-effective diagnostic tool that may provide valuable information about cardiac function and/or indirectly about a person's state (e.g., mental, emotional, blood levels). However, working with ECG data can pose several technical challenges due to the nature of this signal. ECG signals can be easily affected by noise from various sources, including nearby electrical devices and motion artifacts. Even more technically challenging is the variability of the signal: ECG signals can vary significantly for the same individual under slightly different conditions. These technical challenges are exacerbated when trying to infer from ECG indirectly related variables, for example, emotional state or weakly linked physical measurements such a blood glucose level.

Technical challenges such as the aforementioned may be overcome by using large training datasets with labelled data to train machine learning models to obtain sufficient performance. However in practice, many labelled ECG datasets are relatively small, which cannot be used to train machine learning to obtain sufficient performance. In at least some embodiments described herein, training datasets that do not have the relevant labels, which are treated as unlabeled, are used to train a machine learning model(s) to sufficient performance. This unlabeled data may be used to extract meaningful features, which are used for subsequent supervised learning of the primary task, as described herein.

In at least one embodiment, a transfer learning approach is implemented by employing an architecture that couples a first encoder with a second encoder, optionally a convolutional neural network (CNN) with a Transformer. A contrastive learning approaches, for example, as described with reference to [10,3] be used for learning, using self-supervised training from the unlabeled data. In contrast to other self-supervised approaches in time series analysis, where the self-supervised task is frequently based on training the model to recognize signal transformations, such as noise addition, scaling or permutation, for example, as described with reference to [20,8,29,25,19], at least one embodiment of the disclosure takes into account contextual and/or temporal information by masking the ECG input in the latent space.

Experiments performed by Inventors, as described in the Examples section below, indicate that using a training approach according to at least one embodiment described herein greatly improves upon the results of other self-supervised approaches to ECG data analysis. Results are provided for predicting two types of emotional states, as well as for inferring glucose levels based on the ECG signal. Both cases are widely applicable due to the non-invasive, portable nature of ECG, in comparison, for example, to brain-signal-based modalities for estimating emotional states and continuous glucose monitors.

Self-supervised learning is used for learning representations from unlabeled data, for example, as described with reference to [17,4,22,2]. In the context of ECG signal analysis, self-supervised learning may be used for emotion recognition, for example, as described with reference to [20,28,15] and/or other downstream tasks, for example, as described with reference to [29,19,16]. However, previous self-supervised approaches using ECG signals are different than the self-supervised approach of at least one embodiment described herein.

Previous work in ECG representation learning for tasks such as emotion recognition or stress detection has utilized various self-supervised tasks to learn high-level abstract representations from unlabeled ECG data. For example, Sarkar and Etemad [20] apply signal transformations to ECG using operations such as scaling or noise addition and then use transformation recognition as the pretext tasks. In another example, Rabbani and Khan [19] use similar transformations and measure the similarity between different transformed versions of the same segment. In yet another example, Zhang et al [29] flip the original ECG signals horizontally, vertically, or both horizontally and vertically, and then learn to classify between these options. In yet another example, Vazquez-Rodriguez et al [28] propose to take into account contextual information during pre-training, by using a Transformer to reconstruct a masked ECG signal.

In contrast to the aforementioned, at least one embodiment of the disclosure involves a masking process in the latent space and/or does not require multiple hand-crafted transformations. Alternatively or additionally, at least one embodiment uses a two-phase contrastive learning framework as an alternative to traditional methods, such as reconstruction and/or classification objectives. Furthermore, at least one embodiment may extend the envelope of validation by demonstrating the ability to estimate physical states and not just emotional ones.

At least one embodiment described herein relates to mapping an ECG signal to emotional and/or physical states using a 1D-CNN followed by a Transformer. To overcome the limited number of samples, a self-supervised learning scheme that combines masking with a temporal prediction task and/or a matching task, both learned via a contrastive loss, is used. Experiments performed by Inventors, described in the Examples section below, provide evidence that approaches based on at least one embodiment described herein outperform the existing self-supervised approaches for ECG by a wide margin.

At least one embodiment described herein relates to a self-supervised approach for training Transformers on ECG data. Multiple types of pretext tasks are combined. The multiple types of pretext tasks may be shown to be significant in an ablation study conducted by Inventors and described in the Examples section below. The obtained performance surpasses the existing approaches in two challenging supervised learning tasks, obtaining impressive performance despite limited data availability.

At least one embodiment described herein addressed the aforementioned technical problem(s), and/or improves the aforementioned technical field(s), and/or improves upon the aforementioned technical approach(es), by training a machine learning model that includes a first encoder (e.g., 1D CNN) and a second encoder (e.g., transformer) trained on datasets of temporal sequences of physiological measurements of one or more subjects. A baseline latent representation is obtained from the first encoder in response to feeding one of the datasets. A copy latent representation is created as a copy of the baseline latent representation. One or more time-intervals of the copy latent representation are masked. The copy latent representation and the baseline latent representation are included in a training dataset. In a first training phase, the ML model including the first encoder and the second encoder is trained on the training dataset using a self-supervised approach. The ML model may be trained using a temporal objective and/or a contextual contrastive loss objection, optionally using a loss function that includes both objectives. An updated training dataset may be created by including a ground truth label for one or more sample datasets. In a second training phase, the second encoder may be further trained on the updated training dataset using a supervised training approach. The second encoder may be trained while the first encoder is frozen (i.e., not further trained).

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

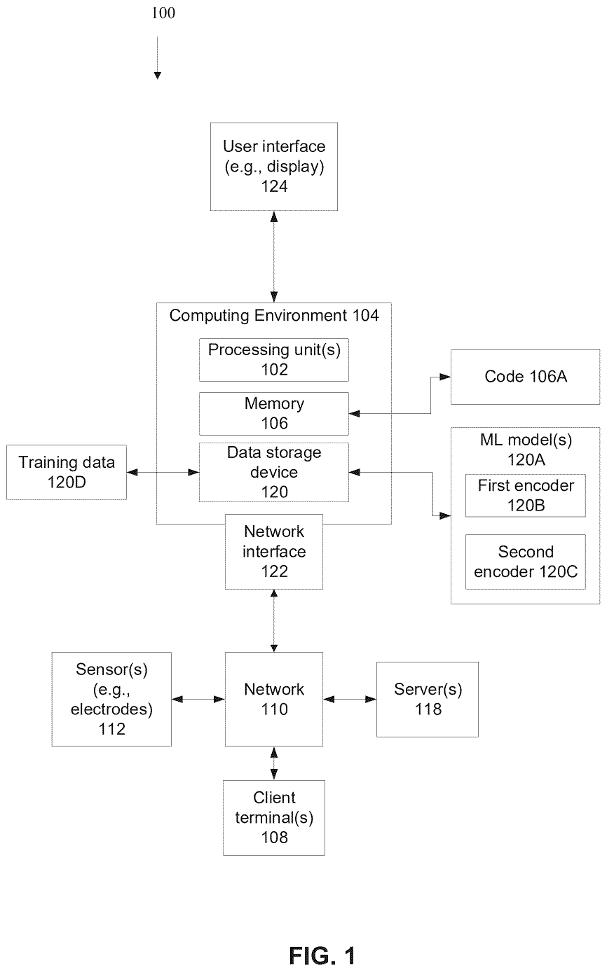

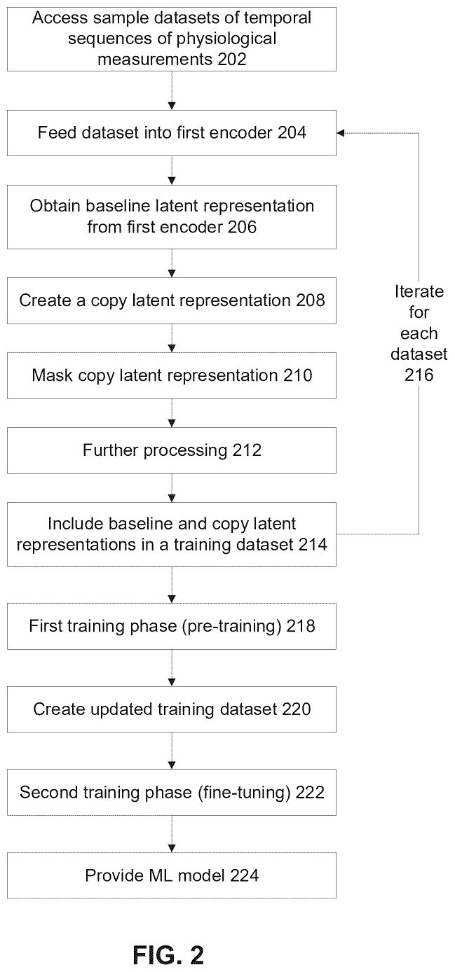

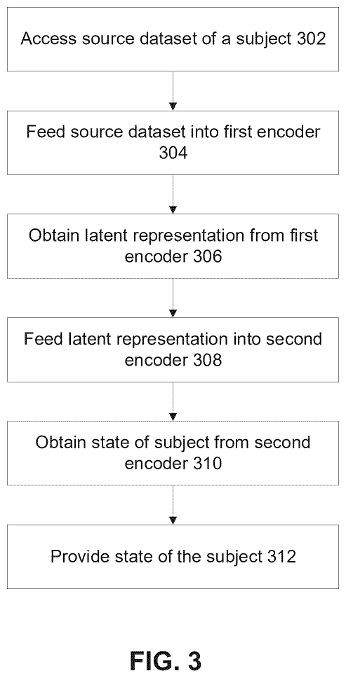

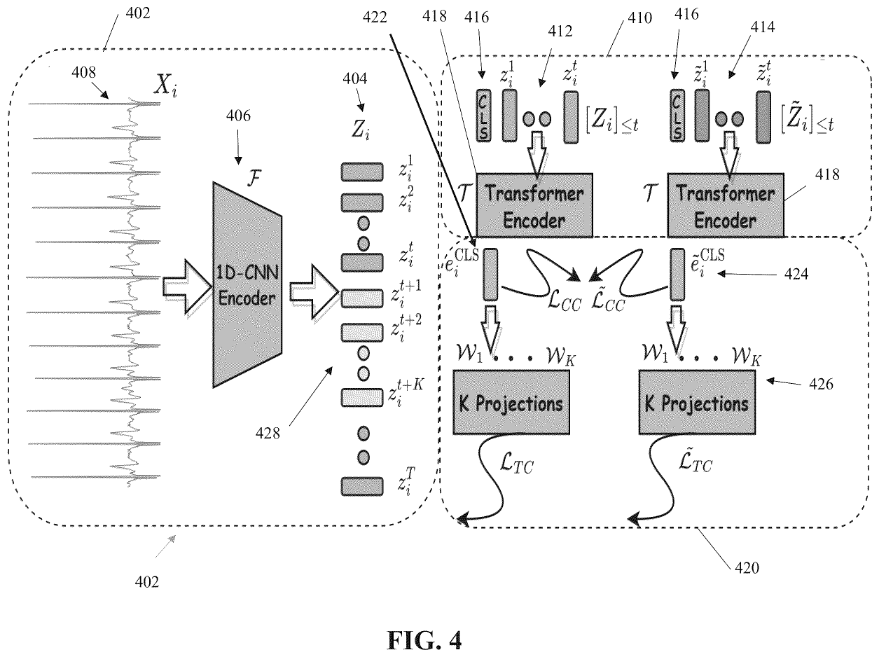

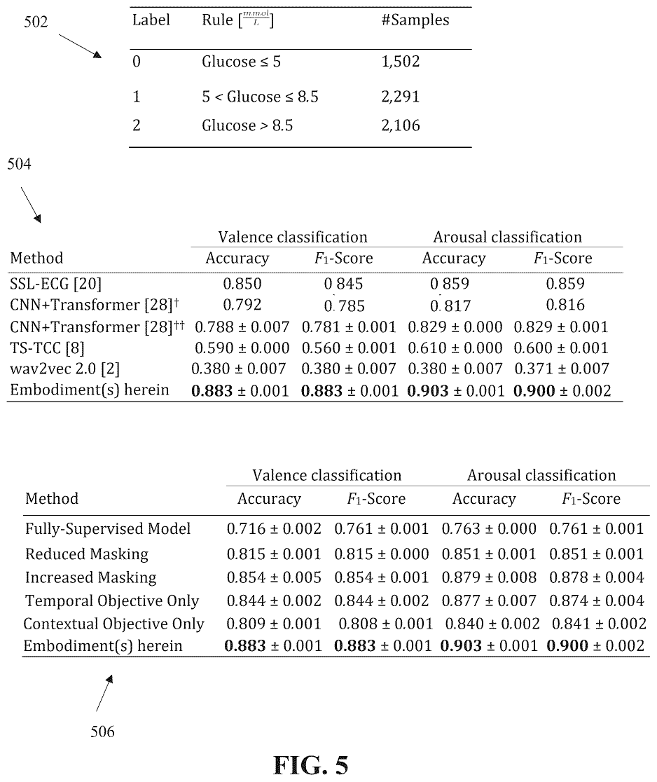

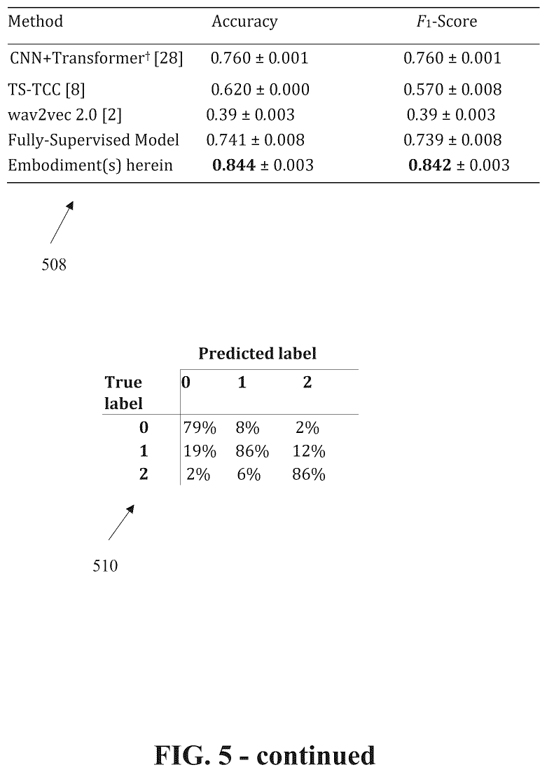

Reference is now made to FIG. 1, which is a block diagram of components of a system 100 for training a machine learning model(s) 120A on a training dataset 120D and/or for inference by the machine learning model(s) 120A, in accordance with some embodiments of the present invention. Reference is also made to FIG. 2, which is a flowchart of a method for training a machine learning model(s) for predicting a state of a subject from a temporal sequence of physiological measurements, in accordance with some embodiments of the present invention. Reference is also made to FIG. 3, which is a flowchart of a method of predicting a state of a subject by a machine learning model(s) fed a temporal sequence of physiological measurements, in accordance with some embodiments of the present invention. Reference is also made to FIG. 4, which is a schematic indicating an exemplary approach for pre-training of a ML model including a first encoder and a second encoder using temporal physiological data, in accordance with some embodiments of the present invention. Reference is also made to FIG. 5, which includes tables 502-510 that present results of the experiments described herein, in accordance with some embodiments of the present invention.

System 100 described with reference to FIG. 1 may implement the features of the method described with reference to FIGS. 2-4, by one or more processors 102 of a computing environment 104 executing code instructions 106A stored on a memory 106.

Computing environment 104 may be implemented as, for example, a client terminal, a server, a virtual machine, a virtual server, a computing cloud, a mobile device, a desktop computer, a thin client, a Smartphone, a Tablet computer, a laptop computer, a wearable computer, glasses computer, and a watch computer.

Computing environment 104 may train ML model 120A using training data 120D obtained from one or more sensor(s) 112. Alternatively or additionally, computing environment 104 may feed input data of a subject obtained from sensor(s) 112 into ML model 120A to obtain a state of a subject, as described herein.

ML model 120A includes a first encoder 120B and a second encoder 120C, optionally a 1D-CNN and a transformer, respectively, as described herein.

Sensor(s) 112 may include one or more electrodes, which may be designed for sensing, for example, ECG, EEG, and/or EMG. Alternatively or additionally, sensor(s) 112 may be designed for sensing a state of a subject and/or enabling the subject to self-label (e.g., self-assessment), which may be used as a ground truth label, for example, an input interface enabling a subject to select the label from multiple candidate labels (e.g., for emotional and/or mental states), a blood glucose level sensor, a blood oxygenation level sensor, and a carbon dioxide blood level sensor.

Multiple architectures of system 100 based on computing environment 104 may be implemented:

In an exemplary implementation of a centralized architecture, computing environment 104 storing code 106A may be implemented as one or more servers (e.g., network server, web server, a computing cloud, a virtual server) that provides centralized services for training ML model(s) 120A, and/or inference of a state of a subject using ML model(s) 120A (e.g., one or more of the acts described with reference to FIGS. 2-4) to one or more servers 118 and/or client terminals 108 over a network 110, for example, providing software as a service (SaaS) to the servers 118 and/or client terminal(s) 108, providing software services accessible using a software interface (e.g., application programming interface (API), software development kit (SDK)), providing an application for local download to the servers 118 and/or client terminal(s) 108, and/or providing functions using a remote access session to the servers 118 and/or client terminal(s) 108, such as through a web browser and/or viewing application. For example, computing environment 104 may train a centralized ML model 120A using data collected from multiple different subjects and/or from different sensor(s) 112. The centralized ML model 120A may be used for centralized inference of a state of a subject using input provided by different client terminals 108 and/or servers 118 which may be obtained from a local sensor(s) 112. In another example, computing environment 104 may provide a centralized service for customized training of ML model 120A, for example, per subject, per sensor(s) 112, per clinic, per hospital, per healthcare facility, and the like.

In an exemplary implementation of a localized architecture, computing environment 104 may be provided dedicated and/or localized and/or customized training of ML model(s) 120A using data obtained from a selected source, for example, of a specific person, a specific group of people, a specific type of sensor, per clinic, per hospital, per healthcare facility, and the like. The customized ML model(s) 120A may be used for inference for the selected source. Customized ML model(s) 120A may be run on computing environment 104, and/or provided to another device for localized inference, for example, to a certain client terminal 108 and/or certain server 118.

Sensor(s) 112 may transmit captured temporal physiological datasets (e.g., of ECG, EEG, EMG signals) to computing environment 104, for example, via a direct connected (e.g., local bus and/or cable connection and/or short range wireless connection), and/or via network 110 and a network interface 122 of computing environment 104 (e.g., where sensor(s) 112 are connected via internet of things (IoT) technology and/or are located remotely from the computing environment). In another implementation, data captured by sensor(s) is sent to computing environment 104 via client terminal 108 and/or server 118 which may be in local communication with sensor(s) 112.

Network interface 122 may be implemented as, for example, a wire connection (e.g., physical port), a wireless connection (e.g., antenna), a network interface card, a wireless interface to connect to a wireless network, a physical interface for connecting to a cable for network connectivity, and/or virtual interfaces (e.g., software interface, application programming interface (API), software development kit (SDK), virtual network connection, a virtual interface implemented in software, network communication software providing higher layers of network connectivity).

Processor(s) 102 of computing environment 104 may be hardware processors, which may be implemented, for example, as a central processing unit(s) (CPU), a graphics processing unit(s) (GPU), field programmable gate array(s) (FPGA), digital signal processor(s) (DSP), and application specific integrated circuit(s) (ASIC). Processor(s) 102 may include a single processor, or multiple processors (homogenous or heterogeneous) arranged for parallel processing, as clusters and/or as one or more multi core processing devices.

Memory 106 stores code instructions 106A executable by hardware processor(s) 102. Exemplary memories 106 include a random access memory (RAM), read-only memory (ROM), a storage device, non-volatile memory, magnetic media, semiconductor memory devices, hard drive, removable storage, and optical media (e.g., DVD, CD-ROM). For example, memory 106 stores code 206A that execute one or more acts of the method described with reference to FIGS. 2-4.

Computing environment 104 may include a data storage device 120 for storing data, for example, ML model(s) 120A (which includes first encoder 120B and second encoder 120C), and/or training data 120D for training ML model(s) 120A. Data storage device 120 may be implemented as, for example, a memory, a local hard-drive, a removable storage unit, an optical disk, a storage device, a virtual memory and/or as a remote server 118 and/or computing cloud (e.g., accessed over network 110).

Computing environment 104 may include and/or are in communication with one or more user interfaces 124 that include a mechanism for inputting data (e.g., self-label collected data) and/or for viewing data (e.g., the state of the subject predicted from the input by the ML model). Exemplary user interfaces 124 include, for example, one or more of, a touchscreen, a display, a keyboard, a mouse, and voice activated software using speakers and microphone.

Referring back to FIG. 2, at 202, multiple sample datasets are accessed. The multiple sample datasets may be of one subject, or multiple subjects.

Each sample dataset includes a temporal sequence of physiological measurements of the respective subject. The temporal sequences of physiological measurements may be obtained from one or more sensors, for example, raw outputs of the sensors and/or computed from measurements made by the sensors. Each temporal sequence may include a continuous recording (which may be sampled into a digital representation) over a selected time interval, for example, about 5 seconds, or 10 seconds, or 30 seconds, or 1 minute, or other time intervals.

Each dataset may include a single-channel signal serving as input. It is to be understood that the single-channel implementation is exemplary and not necessarily limiting, as embodiments described herein may be adapted for multi-channel datasets.

Examples of sample datasets of temporal sequences of physiological measurements include: ECG data from ECG signals obtained by electrodes placed on a chest of the subject, EEG data from EEG signals obtained by electrodes placed on the head (e.g., scalp) of the subject, and EMG data from EMG signals obtained by electrodes placed on the subject according to selected muscles.

In terms of mathematical notation, the dataset may be denoted as Xᵢ∈Rⁿ, where n denotes a multiplicative product of a frequency in Hertz and a length in seconds, and i denotes an index of a sample from which the dataset is extracted.

Some of the sample datasets may be associated with labels, which may be used as ground truth during the self-supervised training phase (i.e., also referred to herein as the second training phase and/or as the fine-tuning training phase). The label may include an emotional state of the subject, for example, anxious, stressed, angry, relaxed, happy, sad, and the like. Alternatively or additionally, the label may indicate a physical state of the subject obtained from measurements made by one or more sensor. Examples of physical states include a blood glucose level, a blood oxygenation level, and a blood carbon dioxide level.

At 204, the sample dataset is fed into a first encoder of a ML model. The first encoder may be implemented as a convolutional neural network (CNN), optionally a one dimensional (1D) CNN.

In terms of mathematical notation, the first encoder, optionally the 1D-CNN, may be denoted F.

An exemplary architecture of the 1D-CNN implementation of the first encoder includes three convolution blocks, a max-pooling layer, and a dropout layer. Each convolution block in the three convolution blocks may include one convolution layer with a Gaussian Error Linear Unit (GELU) [9] activation function. The channel sizes may be for example (32,64,128), and the kernel sizes may be for example (32,16,8). It is noted that other suitable architectures may be implemented.

At 206, an encoding of the sample dataset is obtained as an outcome of the first encoder.

The encoding of the sample dataset generated by the first encoder is also referred to herein as a baseline latent representation.

In terms of mathematical notation, the input Xi is encoded by F. The CNN encoder F is applied to the i-th sample dataset denoted Xi, resulting in a latent representation denoted Zi=F(Xi), where Zi=[zi¹, zi², . . . , zi^{T}].

At 208, a copy latent representation is generated as a copy of the baseline latent representation. It is noted that multiple copies may be created.

In terms of mathematical notation, a copy of Zi, Z^{~}i=[z^{~}i1, z^{~}i2, . . . , z^{~}iT] is created. Zᵢ=[zᵢ¹, zᵢ², . . . , zᵢ^{T}] and Z^{~}_{I} denotes a copy of Zᵢ, Zᵢ=F(Xᵢ).

At 210, one or more time-intervals of the copy latent representation are masked.

As used herein, the terms time-interval and time-step are used interchangeably.

The time-interval(s) may be randomly selected, for example, a random proportion of time-steps is selected from the copy latent representation. The masking may be redone by replacing the data of each selected time-step with zeros.

In terms of mathematical notation, the baseline latent representation (i.e., the unmasked copy) and the masked copy latent representation (i.e., the masked copy) may be respectively denoted as Zᵢ, Z^{~}ᵢ∈R^{m×T}, where m denotes an output dimension of the first encoder and T denotes a number of the time-steps obtained from the original input. Masking may be applied on Zi by selecting a random proportion (denoted p) from its time-steps to be the starting point of a masked span of a length denoted M, and then replacing those spans of time-steps with zeros. The spans may overlap.

At 212, further processing may be performed.

A value (denoted t) may be sampled as a random integer from the interval (0,T−K], where K denotes the number of time-steps selected to predict as a pretext task. The task may be defined to predict, from sequence elements with index [1, . . . , t], the elements with indices [t+1, . . . , t+K].

Two sequences may be generated. The sequences may be generated by cropping the baseline latent representation and the copy latent representation, respectively denoted Zi and Z^{~}I. A classification label, also referred to herein as classification token (denoted CLS), may be added to the beginning of each of the two cropped sequences, mathematically denoted

$${[ {Z}_{i} ]}_{\leq t} = [ CLS , {z}_{i}^{1} , … , {z}_{i}^{t} ] {and [ \tilde{{Z}_{i}} ]}_{\leq t} = [ CLS , {{z}^{∼}}_{i}^{1} , … , {{z}^{∼}}_{i}^{t} ] .$$

The CLS may indicate the ground truth label of the sample dataset.

Each of the two sequences (i.e., the copy and the baseline) may be fed to the second encoder. An embedding of the classification label (CLS token) may be obtained from the second encoder. The embedding of the classification labels obtained from the second encoder may be mathematically denoted as eCLSi=T ([Zi]≤t) and e^{~}CLSi=T ([Z^{~}i]≤t), where e^{CLS}ᵢ, e^{~CLS}ᵢ∈R^{d} and d denotes a dimension of the second encoder.

At 214, the copy latent representation and the baseline latent representation may be included in a training dataset.

At 216, features described with reference to 204-214 may be iterated for each sample dataset, for creating multiple pairs of baseline and copy latent representations.

At 218, a first training phase may be implemented. The first training phase may be referred to as pre-training.

The first training phase may be implemented using a self-supervised training approach.

The pre-training may be performed with a temporal loss objective and/or a contextual contrastive loss objective.

The pre-training may be performed on a machine learning (ML) model that includes the first encoder and a second encoder.

The second encoder may be implemented based on an architecture of a transformer, denoted T. The Transformer, T, may include a dimension of d=128, 2 layers, 4 heads, and a fully-connected layer with the size of d·2=256.

The ML model is trained on the training dataset.

During the first training phase, the ML model may be trained according to a loss function that includes a temporal objective(s). Alternatively or additionally, the ML model may be trained according to a loss function that includes a contextual contrastive loss objective(s)

An exemplary objective function for the first training phase that includes a combination of the temporal objective and the contextual contrastive loss object may be mathematically represented as Lₚᵣₑ₋ₜᵣₐᵢₙ=λ₁(L_{TC}+L^{~}_{TC})+λ₂(L_{CC}+L^{~}_{CC}), where:

λ1 and λ2 are hyperparameters controlling weights and/or significance of the temporal and/or contextual loss terms,

L_{TC}, L^{~}_{TC} denote terms of temporal contrastive loss over representations from the original and masked embeddings, respectively, and

L_{CC}, L^{~}_{CC} denote terms of contextual contrastive loss over representations from the original and masked embeddings, respectively.

The ML model may match different latent representations of the sample dataset using contextual contrasting, such that similarity between the outcome embedding of the classification labels of the two sequences is maximized, and/or similarity with the classification label is minimized.

Alternatively or additionally, a temporal contrastive learning approach may be applied, where the second encoder may predict future time-steps of Zi from a sequence of its own past time-steps, and/or from a sequence of past time-steps of Z^{~}i. The second encoder may match between Zi, Z¹⁸ i in the context of other (e.g., all other) embeddings in the batch.

The ML model may predict one or more future sequence elements, optionally by using a contrastive framework. The contrastive framework may be based on multiple different linear projections (denoted K), mathematically represented as:

Wₖ: R^{d7→m}, project ^{~}e^{CLS}ᵢ and e^{CLS}ᵢ back to the dimension of the latent sequences, each predicting one of the future K≤T−t time-steps of Zᵢ, i.e.,

$$[ {z}_{i}^{t} , … , {z}_{i}^{t + K} ]$$

and Z^{~}_{I}.

The objective is to maximize the dot product between the K projected context vectors and the K matching future time-steps while minimizing the dot product with the timesteps, i.e., those with indices, N_{t,k}=[t+1, . . . , t+k−1, t+k+1, . . . , t+K].

It is noted that loss terms having notations with a tilda (~) refer to calculations over masked embeddings.

The embedding of the classification labels may be projected back to a dimension of the latent representations, each predicting one of the future time intervals of the copy latent representation and/or the baseline latent representation. A dot product between each projected outcome and matching predicted future time interval may be minimized, while minimizing a dot product between each projected outcome and the future time intervals.

The pretext tasks may either use the embedding of the masked sequence or the embedding of the unmasked sequence to predict the future time-steps of the unmasked sequence, for example, using the following mathematical equations:

$${ℒ}_{TC} = - \frac{1}{K} {\sum }_{k = 1}^{K} log \frac{exp ( {( {𝒲}_{k} ( {e}_{i}^{CLS} ) )}^{T} {z}_{i}^{t + K} )}{{\sum }_{n ∈ {𝒩}_{t , k}} exp ( {( {𝒲}_{k} ( {e}_{i}^{CLS} ) )}^{T} {z}_{i}^{n} )}$$

$${\tilde{ℒ}}_{TC} = - \frac{1}{K} {\sum }_{k = 1}^{K} log \frac{exp ( {( {𝒲}_{k} ( {\tilde{e}}_{i}^{CLS} ) )}^{T} {z}_{i}^{t + K} )}{{\sum }_{n ∈ {𝒩}_{t , k}} exp ( {( {𝒲}_{k} ( {\tilde{e}}_{i}^{CLS} ) )}^{T} {z}_{i}^{n} )}$$

Alternatively or additionally to the temporal contrasting, at least one embodiment may use contextual contrasting, in which different encodings of the same original sample are matched. The objective may be to maximize the similarity between the sample denoted e^{CLS}ᵢ and its pair denoted e^{~CLS}ᵢ, while minimizing its similarity with the CLS token of the masked samples of other samples in the batch denoted ^{~}e^{CLS}ⱼ, j/=i, for example,

$${ℒ}_{CC} = - \frac{1}{N} {\sum }_{i = 1}^{N} log \frac{exp ( sim ( {e}_{i}^{CLS} , {\tilde{e}}_{i}^{CLS} ) )}{{\sum }_{j \neq i} exp ( exp ( sim ( {e}_{i}^{CLS} , {\tilde{e}}_{j}^{CLS} ) )}$$

$$\tilde{{ℒ}_{CC}} = - \frac{1}{N} {\sum }_{i = 1}^{N} log \frac{exp ( sim ( {e}_{i}^{CLS} , {\tilde{e}}_{i}^{CLS} ) )}{{\sum }_{j \neq i} exp ( exp ( sim ( {\tilde{e}}_{i}^{CLS} , {e}_{j}^{CLS} ) )}$$

where sim (a,b)=a^{T}b/∥a∥∥b∥ denotes the cosine similarity metric, and N denotes the size of the batch.

Temporal contrasting may use the prediction of K=30 (or other values) future time-steps from K independent projections of the CLS embedding.

At 220, an updated training dataset may be created by including a ground truth label for each sample dataset in the training dataset.

The latent representations sequence Zᵢ=F(Xᵢ) may be obtained, and the CLS token (denoting the ground truth) may be added to the beginning of the latent representations sequence. At 222, a second training phase may be implemented. The second training phase may be referred to as fine-tuning.

The second training phase may be implemented using a supervised training approach.

During the second training phase, the training may be applied to the second encoder, optionally only to the second encoder and not to the first encoder. The first encoder (e.g., CNN) may be frozen. One or more, optionally all, convolutional layers of the first encoder are frozen.

A classification head (denoted C) may be added on top of the second encoder, optionally on top of the output embedding of the CLS token. The classification head may be added to the second encoder for further training for downstream tasks. The addition of the standard classification head may be selected for optimizing the second encoder for the supervised task described herein, which may be specific. The classification head C may be implemented a single linear layer with a size of 128. It is noted that the architecture is exemplary and not necessarily limiting, as other suitable architectures may be implemented.

The second encoder with added classification head may be trained on the updated training dataset using a supervised training approach.

The second encoder may be trained using an objective function denoted as:

L_{fine-tune}=−L_{cce}(yᵢ, C(T[E(Xᵢ)]^{CLS})), where L_{cce} is a softmax function followed by a standard categorical cross-entropy loss and yᵢ∈{1, . . . , c} is an associated label.

At 224, the ML model, which includes the trained first encoder and the trained second encoder, is provided. The ML model generates a prediction of a state of a subject in response to an input of a source dataset of an individual that includes a temporal sequence of physiological measurements of the subject (e.g., measured by one or more sensors).

Referring now back to FIG. 4, a schematic depicting an exemplary approach for the pre-training of the ML model including the first encoder and the second encoder, is depicted. Features described with reference to FIG. 4 may correspond to, and/or may be combined with, and/or replaced with, features described with reference to FIG. 2. Within box 402, a latent sequence 404 denoted Zi is obtained as an outcome of a 1D-CNN encoder 406 denoted F fed a temporal physiological signal 408 denoted Xᵢ, optionally ECG. Within Box 410, sequences of t samples 412 are generated from Zi, as well as a randomly masked clone 414, denoted Zi^{~}, followed by prepending a CLS token 416 to the beginning of each sequence. These sequences are fed into a transformer 418 denoted T. Within box 420, For each sequence 412 and 414, T 418 outputs a context vector 422 and 424 respectively denoted e^{CLS}ᵢ and ^{~}e^{CLS}ᵢ, each is the embedding of the CLS token 416 from each input sequence 412 and 414. Each of these context vectors 422 and 424 is projected 426 K times by W₁, . . . , Wₖ to predict K future time-steps of Zi 428 as shown in box 402, based on optimizing the temporal contrastive objective. In addition, a contextual contrastive loss may be calculated for matching the two CLS tokens in the context of those of other samples.

Referring now back to FIG. 3, at 302, a source dataset of a target subject is accessed. The dataset includes a temporal sequence of physiological measurements of the subject, which may be obtained based on output of one or more sensors, for example, as described herein.

At 304, the source dataset is fed into the first encoder of the ML model. The ML model is trained, for example, as described with reference to FIG. 2.

At 306, a latent representation is obtained from the first encoder.

At 308 the latent representation obtained from the first encoder is fed into the second encoder.

At 310, the state of the subject is obtained as an outcome of the second encoder.

At 312, the state of the subject is provided, for example, presented on a display, stored on a data storage device, forwarded to another computing device, and the like.

Various embodiments and aspects of the present disclosure as delineated hereinabove and as claimed in the claims section below find experimental and/or calculated support in the following examples.

### EXAMPLES

Reference is now made to the following examples, which together with the above descriptions illustrate some embodiments in a not necessarily limiting fashion.

### Data for Pre-Training

Using data from multiple ECG datasets collected via different hardware devices and sampled from diverse populations may help improve the generalization abilities of a machine learning model, for example, as described with reference to [20]. In contrast to approaches described with reference to [20] and [28], the pre-training phase based on at least one embodiment described herein does not contain any data from the dataset that is used for the downstream task, which is likely to make the setting more technically challenging.

The datasets used for pre-training are MAHNOB-HCI (data from channel 2 only) [24], WESAD [21], CASE [23], PsPM-FR [27], PsPM-RRM1-2 [1] and ASCERTAIN [26], from which only data from the left arm is taken, discarding data tagged as low-quality. All datasets are publicly available.

### Evaluation Benchmarks

Experiments were conducted to evaluate training for both emotion recognition and predicting the physical state of a person. First, the pre-trained model is fine-tuned to classify valence and arousal levels. For this, the DREAMER [11] dataset is used, which contains data collected from 23 participants, with 18 different sessions for each participant. Video clips were used with a focus on ECG (and EEG) modalities, and a low-cost wireless portable ECG device with a sampling rate of 256 Hz was used. Self-annotation of the subjects was done using valence, arousal, and dominance. The arousal and valence scores were recorded for the entire session on a scale of 1 to 5, thus level 3 is referred to as the threshold between low and high valence/arousal.

Also described herein is an investigation of the use of the pre-trained model for multi-label classification of blood glucose levels. The model is fine-tuned using the publicly available DINAMO dataset [7], which is a multimodal dataset designed for research on non-invasive management of type 1 diabetes. This dataset includes glucose measurements, ECG, and other physiological signals from 20 healthy subjects and 9 patients with type 1 diabetes. To focus on clinically applicable data, only the data from type-1 diabetes patients was used.

The ECG was monitored with a rate of 250 Hz, and up-sampling to 256 Hz was applied in order to be consistent with the pre-training dataset. The glucose measurements in this dataset were monitored every 5 minutes. To obtain a more frequent reading, linear interpolation was applied, since it is known that changes in glucose are approximately linear on this time scale, for example, as described with reference to [13].

After removing samples that were tagged as low-quality, 5,899 10-second ECG signals remain. The continuous glucose measurements are divided into three clinically relevant levels. Referring now back to FIG. 5, table 502 presents distribution of labels in the glucose dataset.

### Signal Pre-Processing

The signals were filtered with a 4th order Butterworth band-pass filter with a low-cut-off frequency of 0.5 Hz and a high-cut-off frequency of 50 Hz. To make sure that all the datasets have the same frequency as DREAMER, which is 256 Hz, down-sampling was applied on WESAD, CASE and the PsPM datasets to 256 Hz. User-specific z-score normalization, in which signals from each subject have zero-mean and unit variance, was then applied. The signals were segmented without overlapping into a fixed window size of 10 seconds. In total, there were 42,657 10-second segments for the pre-training (all relevant datasets), and 8,372 10-second labeled segments for the fine-tuning from the DREAMER dataset.

### Hyperparameters

During the masking process of the latent representations, a sample of p=0.05 of all time-steps served as starting indices, and the subsequent M=10 time-steps were masked. In the pre-training loss function denoted Lₚᵣₑ₋ₜᵣₐᵢₙ, a slight emphasis is given to the temporal contrasting, with λ₁=1 and λ₂=0.7. In both pre-training and fine-tuning, an Adam optimizer as described with reference to [12] was used with β₁=0.9, β₂=0.99 and a weight decay of 3e⁻⁴. The model was pre-trained for 100 epochs with a learning rate of 3e⁻⁴, and then fine-tuned for 200 epochs with a learning rate of 1e⁻⁵.

### Baselines

The performance of the model trained according to at least one embodiment described herein was compared with the following baselines:
SSL-ECG as described with reference to [20] pre-trains a 1D-CNN model using a self-supervised transformation recognition task to learn representations from 10-second ECG signals. During fine-tuning, a fully-connected network is added to the top of the fixed encoder for the downstream classification task.
CNN+Transformer as described with reference to [28] is based on a 1D-CNN encoder and a Transformer encoder. The model is trained to reconstruct randomly masked samples of a 10-second ECG input. After the pre-training, a fully-connected network is added to the top of the Transformer encoder and the whole model is fine-tuned end-to-end.
TS-TCC as described with reference to [8] is a self-supervised method that pre-trains a 1D-CNN encoder and a Transformer encoder. It applies two different augmentations to a time-series input and then uses it to learn robust temporal relations by its proposed temporal and contextual contrasting framework. After pre-training, the CNN encoder is optimized for a specific downstream classification task.
wav2vec 2.0 as described with reference to [2] is a speech recognition model that uses unsupervised pre-training on large amounts of unlabeled audio data. It learns to predict masked portions of audio waveforms, which helps it encode the underlying phonetic information present in speech signals. The learned representations can be generalized across a wide range of speech-related tasks, and sometimes even to other domains [5,18,14].

Results of a few ablations are described herein. The Fully-Supervised Model is trained end-to-end without pre-training. Reduced Masking is an ablation where a much smaller amount of embeddings are masked, by keeping p=0.05 and reducing the span by setting M=2. Conversely, the Increased Masking model masks a much bigger amount of embeddings, by keeping p=0.05 and increasing the span to M=25. The model Temporal Objective Only is pre-trained with λ₁=1, λ₂=0, thus Lₚᵣₑ₋ₜᵣₐᵢₙ=L_{TC}+L^{~}_{TC}. Conversely, Contextual Objective Only has λ₁=0, λ₂=1, i.e., Lₚᵣₑ₋ₜᵣₐᵢₙ=L_{CC}+L^{~}_{CC}.

### Results

### Valence and Arousal Classification.

A 10-fold cross-validation approach was used to evaluate approaches based on at least one embodiment described herein. 90% of the data was randomly selected for training and the remaining 10% was used for testing. The mean accuracy and mean F1-score between positive and negative classes of valence/arousal were used as metrics. The confidence intervals were calculated using the critical value of Student's t-distribution with 9 degrees of freedom.

Referring now back to FIG. 5, table 504 presents valence and arousal classification results on the DREAMER dataset. Table 504 presents the comparison to the baseline. Evidently, approaches based on at least one embodiment described herein outperforms previous self-supervised ECG emotion classification methods described with reference to [20, 28]. It may also be observed that pre-training the model presented in [8] and applying their self-supervised method on the ECG pre-training dataset disclosed herein results in low performance when fine-tuning for downstream tasks. In addition, fine-tuning approaches described with reference to [2] on ECG data does not seem to be a good choice.

### Ablation Study

Referring now back to FIG. 5, table 506 presents an ablation study conducted on the DREAMER dataset. Table 506 presents the comparison to the baseline. Table 506 presents the ablation analysis performed in the context of emotion recognition with the goal of exploring the importance of each component of approaches based on at least one embodiment described herein. Since the performance of the fully supervised model is far lower than that of the full method, it provides evidence evident that the pre-training phase is significant. When one of the two types of contrastive loss (temporal or contextual) is removed, performance drops. Out of the two, the temporal objective seems to be more beneficial. Reducing the amount of masking greatly decreases performance. Excessive masking may also detrimental, but to a slightly lesser extent for the explored set of parameters.

### Glucose Level Estimation

Referring now back to FIG. 5, table 508 present results of glucose classification, where the same 10-fold cross-validation approach was used to evaluate approaches based on at least one embodiment described herein on multiclass glucose classification. Also provided herein are the results for an ablation of the Fully-Supervised Model, where the whole model is trained end-to-end for glucose classification without the pre-training phase described herein. Based on table B08, the model trained according to at least one embodiment described herein outperforms all baselines by a sizeable gap. Interestingly, the confusion matrix, presented in table 510, shows that the distinctive hypoglycemia and hyperglycemia classes are seldom confused.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

It is expected that during the life of a patent maturing from this application many relevant machine learning models will be developed and the scope of the term machine learning model is intended to include all such new technologies a priori.

As used herein the term “about” refers to ±10%.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

1. Bach, D. R., Gerster, S., Tzovara, A., Castegnetti, G.: PsPM-RRM1-2: SCR, ECG, respiration and eye tracker measurements in response to electric stimulation or visual targets (September 2019). www(dot)doi(dot)org/10.5281/zenodo.4287111, www(dot)doi(dot)org/10.5281/zenodo.4287111, See the readme file for more detail. This version fixes incorrect SCR scaling in one participant.
2. Baevski, A., Zhou, Y., Mohamed, A., Auli, M.: wav2vec 2.0: A framework for self-supervised learning of speech representations. Advances in neural information processing systems 33, 12449-12460 (2020)
3. Chen, T., Kornblith, S., Norouzi, M., Hinton, G.: A simple framework for contrastive learning of visual representations. In: III, H. D., Singh, A. (eds.) Proceedings of the 37th International Conference on Machine Learning. Proceedings of Machine Learning Research, vol. 119, pp. 1597-1607. PMLR (13-18 Jul. 2020), www (dot) proceedings (dot) mlr (dot) press/v119/chen20j(dot)html
4. Chen, X., Xie, S., He, K.: An empirical study of training self-supervised vision transformers. In: Proceedings of the IEEE/CVF International Conference on Computer Vision. pp. 9640-9649 (2021)
5. Conneau, A., Baevski, A., Collobert, R., Mohamed, A., Auli, M.: Unsupervised cross-lingual representation learning for speech recognition. arXiv preprint arXiv: 2006.13979 (2020)
6. Devlin, J., Chang, M. W., Lee, K., Toutanova, K.: Bert: Pre-training of deep bidirectional transformers for language understanding. arXiv preprint arXiv: 1810.04805 (2018)
7. Dubosson, F., Ranvier, J. E., Bromuri, S., Calbimonte, J. P., Ruiz, J., Schumacher, M.: The open dinamo dataset: A multi-modal dataset for research on non-invasive type 1 diabetes management. Informatics in Medicine Unlocked 13, 92-100 (2018). www(dot)doi(dot)org/10.1016/j.imu.2018.09.003, www (dot) sciencedirect (dot) com/science/article/pii/S2352914818301059
8. Eldele, E., Ragab, M., Chen, Z., Wu, M., Kwoh, C. K., Li, X., Guan, C.: Selfsupervised contrastive representation learning for semi-supervised time-series classification. arXiv preprint arXiv: 2208.06616 (2022)
9. Hendrycks, D., Gimpel, K.: Gaussian error linear units (gelus). arXiv preprint arXiv: 1606.08415 (2016)
10. Jaiswal, A., Babu, A. R., Zadeh, M. Z., Banerjee, D., Makedon, F.: A survey on contrastive self-supervised learning. Technologies 9(1), 2 (2020)
11. Katsigiannis, S., Ramzan, N.: Dreamer: A database for emotion recognition through EEG and ECG signals from wireless low-cost off-the-shelf devices. IEEE Journal of Biomedical and Health Informatics 22(1), 98-107 (2018). www(dot)doi(dot)org/10.1109/JBHI.2017.2688239
12. Kingma, D. P., Ba, J.: Adam: A method for stochastic optimization. arXiv preprint arXiv: 1412.6980 (2014)
13. Li, J., Tobore, I., Liu, Y., Kandwal, A., Wang, L., Nie, Z.: Non-invasive monitoring of three glucose ranges based on ECG by using DBSCAN-CNN. IEEE Journal of Biomedical and Health Informatics 25(9), 3340-3350 (2021). www(dot)doi(dot)org/10.1109/JBHI.2021.3072628
14. Millet, J., Caucheteux, C., Orhan, P., Boubenec, Y., Gramfort, A., Dunbar, E., Pallier, C., King, J. R.: Toward a realistic model of speech processing in the brain with self-supervised learning. arXiv preprint arXiv: 2206.01685 (2022)
15. Montero Quispe, K. G., Utyiama, D. M., Dos Santos, E. M., Oliveira, H. A., Souto, E. J.: Applying self-supervised representation learning for emotion recognition using physiological signals. Sensors 22(23), 9102 (2022)
16. Oh, J., Chung, H., Kwon, J. m., Hong, D. g., Choi, E.: Lead-agnostic self-supervised learning for local and global representations of electrocardiogram. In: Conference on Health, Inference, and Learning. pp. 338-353. PMLR (2022)
17. Oord, A. v. d., Li, Y., Vinyals, O.: Representation learning with contrastive predictive coding. arXiv preprint arXiv: 1807.03748 (2018)
18. Pan, X., Chen, P., Gong, Y., Zhou, H., Wang, X., Lin, Z.: Leveraging uni-modal self-supervised learning for multimodal audio-visual speech recognition. arXiv preprint arXiv: 2203.07996 (2022)
19. Rabbani, S., Khan, N.: Contrastive self-supervised learning for stress detection from ECG data. Bioengineering 9(8), 374 (2022)
20. Sarkar, P., Etemad, A.: Self-supervised ECG representation learning for emotion recognition. IEEE Transactions on Affective Computing 13(3), 1541-1554 (2020)
21. Schmidt, P., Reiss, A., Duerichen, R., Marberger, C., Van Laerhoven, K.: Introducing wesad, a multimodal dataset for wearable stress and affect detection. In: Proceedings of the 20th ACM International Conference on Multimodal Interaction. p. 400-408. ICMI '18, Association for Computing Machinery, New York, NY, USA (2018). www(dot)doi(dot)org/10.1145/3242969.3242985, www(dot)doi(dot)org/10.1145/3242969.3242985
22. Schneider, S., Baevski, A., Collobert, R., Auli, M.: wav2vec: Unsupervised pretraining for speech recognition. arXiv preprint arXiv: 1904.05862 (2019)
23. Sharma, K., Castellini, C., van den Broek, E. L., Albu-Schaeffer, A., Schwenker, F.: A dataset of continuous affect annotations and physiological signals for emotion analysis. Scientific data 6(1), 196 (2019)
24. Soleymani, M., Lichtenauer, J., Pun, T., Pantic, M.: A multimodal database for affect recognition and implicit tagging. IEEE Transactions on Affective Computing 3(1), 42-55 (2012). www(dot)doi(dot)org/10.1109/T-AFFC.2011.25
25. Soltanieh, S., Etemad, A., Hashemi, J.: Analysis of augmentations for contrastive ecg representation learning. In: 2022 International Joint Conference on Neural Networks (IJCNN). pp. 1-10. IEEE (2022)
26. Subramanian, R., Wache, J., Abadi, M. K., Vieriu, R. L., Winkler, S., Sebe, N.: Ascertain: Emotion and personality recognition using commercial sensors. IEEE Transactions on Affective Computing 9(2), 147-160 (2018). www(dot)doi(dot)org/10.1109/TAFFC.2016.2625250
27. Tzovara, A., Bach, D. R., Castegnetti, G., Gerster, S., Hofer, N., Khemka, S., Korn, C. W., Paulus, P. C., Quednow, B. B., Staib, M.: PsPM-FR: SCR, ECG and respiration measurements in a delay fear conditioning task with visual CS and electrical US. (August 2018). www(dot)doi(dot)org/10.5281/zenodo.1405394, www(dot)doi(dot)org/10.5281/zenodo.1405394
28. Vazquez-Rodriguez, J., Lefebvre, G., Cumin, J., Crowley, J. L.: Transformer-based self-supervised learning for emotion recognition. In: 2022 26th International Conference on Pattern Recognition (ICPR). pp. 2605-2612. IEEE (2022)
29. Zhang, W., Geng, S., Hong, S.: Learning ECG representations based on manipulated temporal-spatial reverse detection. arXiv preprint arXiv: 2202.12458 (2022)

1. A method of predicting a state of a subject, comprising:
feeding a dataset including a temporal sequence of physiological measurements of the subject into a machine learning (ML) model comprising a first encoder and a second encoder; and
obtaining the state of the subject as an outcome of the second encoder,
wherein the ML model is trained by:
for each sample dataset of a plurality of sample datasets, wherein each sample dataset includes a sample temporal sequence of physiological measurements of the subject:
feeding the sample dataset into the first encoder,
obtaining a baseline latent representation as an outcome of the first encoder,
creating at least one copy latent representation from the baseline latent representation,
masking at least one time-interval of each at least one copy latent representation, and
including the at least one copy latent representation and the baseline latent representation in a training dataset;
in a first training phase, training the ML model including the first encoder and the second encoder on the training dataset using a self-supervised approach;
creating an updated training dataset by including a ground truth label for each sample dataset in the training dataset; and
in a second training phase, further training the second encoder on the updated training dataset using a supervised training approach.

2. The method of claim 1, wherein the ML model generates a prediction of a state of a subject from an input of a source dataset of an individual.

3. The method of claim 1, further comprising:
feeding the dataset into the first encoder to obtain a latent representation;
feeding the latent representation into the second encoder; and
obtaining the prediction of the state of the subject from the second encoder.

4. The method of claim 1, further comprising during the second training phase: freezing at least one convolutional layer of the first encoder and/or freezing the first encoder, and adding a classification head on top of an output embedding of the ground truth.

5. The method of claim 1, wherein the first encoder is implemented as a one dimensional (1D) convolutional neural network CNN).

6. The method of claim 5, wherein the first encoder includes three convolution blocks, a max-pooling layer, and a dropout layer, and wherein each convolution block in the three convolution blocks comprises one convolution layer with a Gaussian Error Linear Unit (GELU) activation function.

7. The method of claim 1, wherein the second encoder is implemented as a transformer.

8. The method of claim 1, wherein during the first training phase, the ML model is trained
according to a loss function comprising at least one temporal objective.

9. The method of claim 1, wherein during the first training phase, the ML model is trained
according to a loss function comprising at least one contextual contrastive loss objective.

10. The method of claim 1, further comprising, for each sample dataset:
generating two sequences by cropping the at least one copy latent representation and the baseline latent representation, and adding a classification label indicating the ground truth label of the sample dataset; and
feeding each of the two sequences to the second encoder for obtaining outcome embedding of the classification label,
wherein during the first training phase the ML model further matches different latent representations of a sample dataset using contextual contrasting, such that at least one of: similarity between the outcome embedding of the classification labels of the two sequences is maximized, and similarity with the classification label is minimized.

11-12. (canceled)

13. The method of claim 10, wherein the first training phase comprises predicting by the ML model, at least one future sequence element by using a contrastive framework with a plurality of different linear projections, projecting the outcome embedding of the classification labels back to a dimension of the latent representations each predicting one of the future time intervals of the at least one copy latent representation and/or the baseline latent representation, and minimizing a dot product between each projected outcome and matching predicted future time interval while minimizing a dot product between each projected outcome and the future time intervals.

14-15. (canceled)

16. The method of claim 1, wherein the dataset includes at least one of: ECG data from ECG signals obtained by electrodes on the subject, EEG data from EEG signals obtained by electrodes on the subject, EMG data from EMG signals obtained by electrodes on the subject.

17. The method of claim 16, wherein the dataset comprises a single-channel signal input.

18. (canceled)

19. The method of claim 1, wherein the state of the subject comprises a physical state, wherein the ground truth of the physical state is obtained from measurements made by a sensor.

20. The method of claim 19, wherein the physical state is selected from: blood glucose level, blood oxygenation level, blood carbon dioxide level.

21. The method of claim 1, wherein the dataset is denoted as Xᵢ∈Rⁿ, where n denotes a multiplicative product of a frequency in Hertz and a length in seconds, and i denotes an index of a sample from which the dataset is extracted.

22. The method of claim 1, wherein the baseline latent representation and the masked copy latent representation are respectively denoted as Zᵢ, Z^{~}ᵢ∈R^{m×T}, wherein m denotes an output dimension of the first encoder and T denotes a number of the time-steps obtained from the input.

23. The method of claim 1, wherein masking comprises selecting a random proportion from a plurality of time-steps to be a starting point of a masked span of a selected length, and replacing each of the plurality of time-steps with zeros.

24. A method of training a machine learning (ML) model, comprising:
for each sample dataset of a plurality of sample datasets, wherein each sample dataset includes a temporal sequence of physiological measurements of the subject:
feeding the sample dataset into a first encoder,
obtaining a baseline latent representation as an outcome of the first encoder,
creating at least one copy latent representation from the baseline latent representation,
masking at least one time-interval of each at least one copy latent representation, and
including the at least one copy latent representation and the baseline latent representation in a training dataset;
in a first training phase, training the ML model including the first encoder and a second encoder on the training dataset using a self-supervised approach;
creating an updated training dataset by including a ground truth label for each sample dataset in the training dataset; and
in a second training phase, further training the second encoder on the updated training dataset using a supervised training approach.

25. A system for predicting a state of a subject, comprising:
at least one processor executing a code for:
feeding a dataset including a temporal sequence of physiological measurements of the subject into a machine learning (ML) model comprising a first encoder and a second encoder; and
obtaining the state of the subject as an outcome of the second encoder,
wherein the ML model is trained by:
for each sample dataset of a plurality of sample datasets, wherein each sample dataset includes a sample temporal sequence of physiological measurements of the subject:
feeding the sample dataset into the first encoder,
obtaining a baseline latent representation as an outcome of the first encoder,
creating at least one copy latent representation from the baseline latent representation,
masking at least one time-interval of each at least one copy latent representation, and
including the at least one copy latent representation and the baseline latent representation in a training dataset;
in a first training phase, training the ML model including the first encoder and the second encoder on the training dataset using a self-supervised approach;
creating an updated training dataset by including a ground truth label for each sample dataset in the training dataset; and
in a second training phase, further training the second encoder on the updated training dataset using a supervised training approach.
